(19) United States
(12) Patent Application Publication
(10) Pub. No.: US 2010/0256610 A1
(43) Pub. Date: Oct. 7, 2010

(54) APPARATUS AND METHOD OF DETECTION AND LOCALIZED TREATMENT OF ABNORMAL CONDITIONS

(76) Inventor: Basil Rigas, Stony Brook, NY (US)

Correspondence Address: THE FARRELL LAW FIRM, LLP, 290 Broadhollow Road, Suite 210E, Melville, NY, 11747, US

(21) Appl. No.: 12/739,917

(22) PCT Filed: Oct. 27, 2008
(86) PCT No.: PCT/US08/81368

§ 371 Date: Apr. 26, 2010

Related U.S. Application Data
(60) Provisional application No. 60/982,555, filed on Oct. 25, 2007.
(60) Provisional application No. 61/108,061, filed on Oct. 24, 2008.

(51) Int. Cl. A61M 5/00

(52) U.S. Cl. 604/891.1

(57) ABSTRACT

A method and apparatus for early detection and treatment of abnormal biological activity by specific, localized therapeutic response based on biomarker detection to minimize levels of administrated drugs and reducing toxicity. An external communication device communicates with an implanted probe calibrated to detect a biomarker and an implanted therapeutic delivery system.

### PRIORITY

This application claims priority to provisional application Ser. No. 60/982,555, filed with the U.S. Patent and Trademark Office on Oct. 25, 2007, to provisional application Ser. No. 61/108,061, filed with the U.S. Patent and Trademark Office on Oct. 24, 2008, the contents of each of which are incorporated herein by reference.

### BACKGROUND

The present invention relates to a method and apparatus for the early detection and treatment of manifestations of human and animal diseases, including but not limited to cancer, cardiovascular diseases, neurological, intestinal, lung, skin and kidney abnormalities.

Various chemical compound biomarkers have been developed to detect disease and other indicators of an individual's health. As described in WO 2005/003716 A2, U.S. Pat. No. 6,059,724, and Pub No. U.S. 2003/0228640 A1, the disclosure of each of which is incorporated herein by reference, biomarkers are typically used to determine, on a post hoc basis, a condition that a person suffers from, including early changes of a disease before it is fully expressed clinically.

The detection of early manifestations of cancer, i.e., early neoplastic changes, represents an illustrative example. Conventional cancer prevention strategies utilize biomarker detection via blood analysis to determine when to deliver therapeutic or preventive drugs to kill transformed cells. However, most biomarkers are only detectable in blood when the biomarkers exist in relatively large quantities. Such large quantities are manufactured by tumors when the tumors have already grown to a relatively large size.

As cancer cells become malignant, the cells gradually develop increasingly complex defenses against the body's immune and other anti-tumor defense systems. These defenses are also effective against drug treatments, making it vital to detect and kill abnormal cells at the earliest possible moment. Many drugs used to treat cancer carry severe, sometimes even fatal, side effects. At times such drugs can be administered in amounts that are not fully effective, even at relatively high doses. Such conventional systems cannot, however, provide a targeted, localized response to an existing condition.

Moreover, conventional systems typically provide only non-specific indications of an adverse health occurrence. Treatment of such non-specific indications typically involves administration of excess amounts of therapeutic pharmaceuticals, to ensure treatment at locations that are often remote from the point at which the pharmaceuticals are administered.

Recently, advances have been made in developing a more precise method to measure whether chemotherapy has reached a tumor by affixing a miniaturized silicon sensor to a cancerous tumor. See, Fargen, J., Tiny Implants Could Offer Huge Insights On Cancer, Boston Herald, Jan. 14, 2007, page 6. However, conventional systems and methods fail to utilize biomarker(s) and also fail to administer a therapeutic treatment at a predefined, specific location, in a timely manner.

The present invention allows for cancer and other abnormal conditions representing pre-cancerous stages, by detection of biomarkers in minute quantities directly at the site of their production, and release of therapeutic agents at the same site. The present invention minimizes toxicity by using a sensor to detect the abnormality and focus drug delivery to a location within the body where the neoplastic changes, including but not limited to benign and malignant cancerous changes, are present. The present invention is also applicable to cardiovascular diseases, lung diseases, kidney diseases, brain diseases, and skin diseases, but is not limited to such specific ailments.

### SUMMARY OF THE INVENTION

The present invention overcomes the above-described shortcoming of conventional systems by providing an apparatus and method for early and localized detection that also allows for treatment with minimal amounts of drugs delivered locally to the area of neoplastic change, thereby reducing drug toxicity.

In the present invention, a method and apparatus are provided to detect and provide targeted treatment of abnormal conditions, including cancer. In the present invention, an external communication device is provided for communicating with an implantable probe calibrated to detect a biomarker and for communicating with an implantable therapeutic delivery system that, upon detection by the probe of the biomarker, delivers a therapeutic agent at the predefined location for maximum effectiveness in treatment with minimal or no side effects.

The present invention provides early detection and treatment of cancer and other abnormal conditions by biomarker detection followed by specific and immediate therapeutic response. A preferred embodiment of the present invention prevents development and/or recurrence of cancer by use of wireless sensors for communication with a detection probe and local therapeutic delivery agent.

### BRIEF DESCRIPTION OF THE DRAWINGS

The above and other objects, features and advantages of certain exemplary embodiments of the present invention will be more apparent from the following detailed description taken in conjunction with the accompanying drawings, in which:

FIG. 1 shows a general arrangement of a patient having an embodiment of the present invention affixed to an internal organ, also shown an external communication device for remote sensing;

FIG. 2 is an organ onto which a probe sensor and therapeutic delivery system of the present invention have been affixed;

FIG. 3 shows a preferred unitary arrangement of a probe sensor, a communication component for the sensor and a therapeutic delivery system of the present invention;

FIG. 4 illustrates application of the present invention for prevention of colon cancer; and

FIG. 5 illustrates application of the present invention for prevention of breast cancer.

### DETAILED DESCRIPTION OF THE PREFERRED EMBODIMENTS

The following detailed description of preferred embodiments of the invention will be made in reference to the accompanying drawings. In describing the invention, explanation about related functions or constructions known in the art are omitted for the sake of clarity in understanding the concept of the invention, to avoid obscuring the invention with unnecessary detail. The description provided herein will often utilize cancer as an example of an abnormal condition that can be diagnosed and/or treated by the present invention. However, the present invention is not limited to cancer diagnosis and treatment.

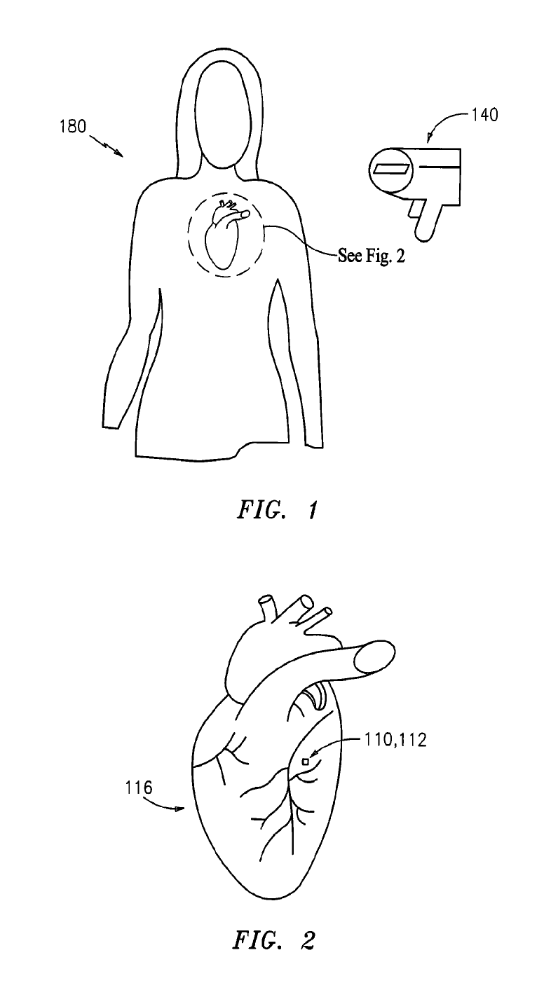

The present invention provides for automated detection and provides targeted treatment of various diseases, utilizing an external communication device 140, an implantable probe 110 calibrated to detect one or more biomarkers 130, as shown in FIGS. 1-3. The communication device 140 wirelessly communicates with the implantable probe 110 as well as with an implantable therapeutic delivery system 120 that preferably is fixedly positioned within a patient's body at a site where release of the therapeutic agent will have maximum impact.

The external communication device, which preferably operates as a dedicated short-range communication system such as a Radio Frequency Identification (RFID) communication system, includes an antenna/transceiver tuned to a common frequency to facilitate information exchange. The implantable probe is queried via radio frequency signal, which transfers information regarding biosensor activity to the external communication device. When configured as an RFID tag, an output indicative of biosensor activity changes, preferably in a manner described by U.S. Pat. No. 6,720,866, to Sorrells et al., the disclosure of which is incorporated herein by reference.

A preferred embodiment of the present invention includes an active RFID 114 that changes informational state upon detection of a target biomarker, thereby allowing for remote monitoring. Patients who previously had cancer are often prone to recurrence. For these and other patients, the remote monitoring via RFID allows immediate, non-intrusive observations of whether a biomarker activity to be obtained, which greatly improves the patient's sense of wellness.

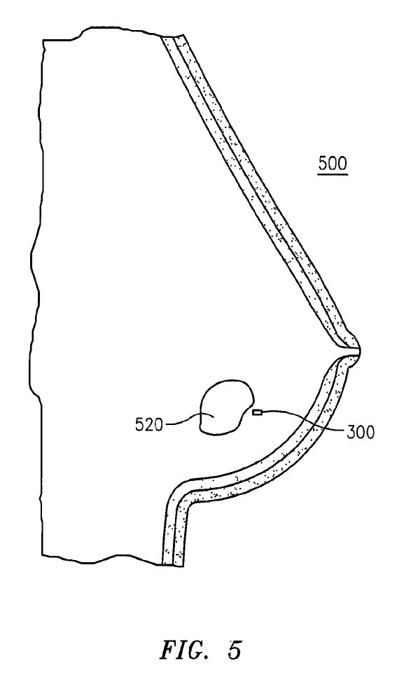

The present invention provides for communication between the implantable probe 110, the external communication device 140, and the implantable therapeutic delivery system 120 is performed without the need for a biopsy or other invasive procedure. Such communication with an external monitoring and processing device 300 (FIG. 4) allows for a physician and other specialized caregivers to provide a complete and up to date assessment of patient health based on determined biomarker activity, combined with other factors external to the location at which the probe is implanted. Such other factors that are considered can include administration of other medications to the patient in which the probe 110 is implanted.

The external monitoring and processing device 300 will provide instruction to the implantable therapeutic delivery system 120 regarding timing and amount of release of therapeutic agent corresponding the sensed biomarker. That is, upon sensing a change in the tissue, e.g. heart, lung, brain, etc., onto which the sensor 112 is affixed, the measured change is wirelessly transmitted to the external communication device 140 for processing at the external monitoring and processing device 300, which makes a determination of a proper amount and timing of release of the therapeutic agent.

In a preferred embodiment, a computer repository is provided that includes algorithms for fast processing of sensor readouts to provide immediate determination based on biomarker detection, particularly when a probe include a plurality of biomarkers. To expedite detection, a patient who has an implanted probe is preferably provided with a portable RFID interrogator that communicates via telephone with the repository, which is located in a remote location.

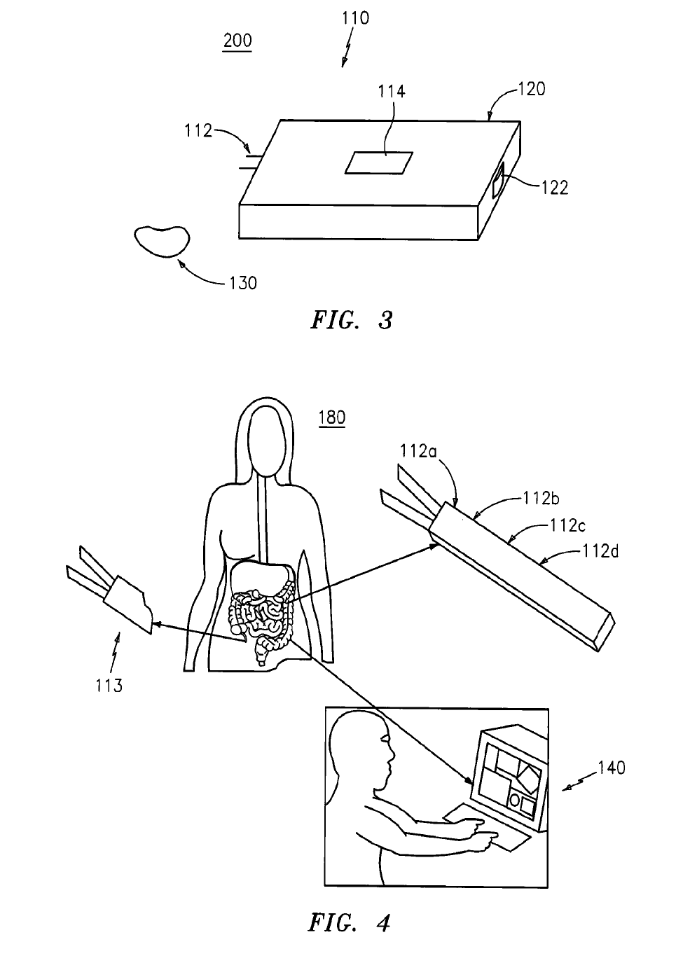

Biopsies are often performed to either assess the status of the tissue of interest or to confirm an anticipated abnormal condition, such as prostate or breast cancer. It is preferred that such conditions be continually monitored after the initial biopsy. In a preferred embodiment of the present invention, a unitary arrangement of a probe sensor, a communication component for the sensor and a therapeutic delivery system 200 (FIG. 3) is fixed within the patient's body at the time of the initial biopsy.

In the present invention, the sensor 112 is fixedly positioned within the patient at a predetermined location suspected to be a source of cancer or other abnormal biological function senses a biological signal. In a preferred embodiment, the sensor is implanted during surgery after removal of a cancerous growth, with the surgeon preferably placing the sensor 112 within the void created by removal of the cancerous tissue. The sensor 112 is fixed to a site of known abnormal biological activity, where biomarkers will typically be produced should the abnormal biological activity reappear. Implanting the sensor 112 at the location of a prior abnormal biological activity allows the patient to obtain exact measurements of the amounts of biomarkers being produced. Sensing at the site of biomarker production, rather than in the bloodstream, which can dilute the biomarker, improves accuracy.

In a preferred embodiment, an assembly combining the sensor 112 and therapeutic delivery system 120 is affixed within the patient to allow for local, in situ, delivery of the therapeutic agent at the site of any nascent tumors, which often develop at locations where tumors were removed, thereby significantly reducing risk of recurrence.

As shown in FIG. 5, the external monitoring and processing device 300 is readily implantable in the fatty regions of the human breast 500. The external monitoring and processing device 300 is preferably implanted at the time a biopsy is conducted when a lump is detected by external self-examination or examination by a health professional. If the lump develops into a cancerous tumor 520, the external monitoring and processing device 300 will recognize the associated biomarkers, will release appropriate therapeutic agents, and will send via the probe sensor 112 an indication of biomarker detection for analysis and potential additional treatment.

Cancer is not the only abnormal health condition characterized by markers in mammalian and non-mammalian patients. Other diseases, such as heart attacks, can be detected and treated utilizing the present invention. For example, by the formation of a clot is characterized by release of enzymes indicating cardiac damage or markers indicating formation of a clot. The present invention senses the enzymes/biomarkers, as well as changes therein, such as changes in concentration, and releases an anti-clotting drug to save a patient's life. In a preferred embodiment of the present invention, a change in anticipated biomarker activity is monitored to detect a change in either an amount or activity of the biomarker. Biomarkers are often proteins having a specific function or activity, for example amylase, which functions to break starch into sugar. In a preferred embodiment of the present invention, an amount of amylase is detected, rather than how much starch is acted upon.

Numerous organs include sufficient space within which the probe 110 and therapeutic delivery system 120 can be affixed. For example, the lumen of the large bowel, the vicinity of the prostate, the soft, fatty tissue of the human breast, and the branches of the trachea inside the lung, within which many human cancers grow, the myocardium or vessels, of particular interest those vessels draining an area of interest, all provide sufficient space to house the probe 110 and therapeutic delivery system 120.

In a preferred embodiment, the detection probe is provided as a nanosensor implanted within a body part believed to be susceptible to cancer, to immediate detect relevant biomarkers. Upon detection of an abnormal amount of cancer biomarker, minute amounts of an appropriate drug are delivered locally to eliminate the neoplastic cells. The present invention provides an advantage of elimination of the neoplastic cells at their nascent stage, allowing for use of a markedly reduced amount of therapeutic agent. The present invention links biomarkers with a localized therapeutic drug delivery system via wireless transmission, thereby providing focused cancer care without repeated invasive procedures.

In a preferred embodiment, the implantable probe includes a prostate cancer marker such as the biomarker described in Pub. No. U.S. 2003/0228640 A1 of Getzenberg, the disclosure of which is incorporated herein by reference. The implantable probe is fixed in a position near or inside the prostate gland and monitors biological changes to detect any abnormal formations in the prostate. Getzenberg notes that certain biomarkers are present in normal tissue adjacent to the tumor tissue in individuals having prostate cancer, but is absent in the prostate of individuals without the disease. The present invention utilizes a detection of such biomarkers to release, at a localized application point, an effective amount of a therapeutic drug. Because the therapeutic is locally released, efficacy greatly improves. Moreover, a preferred embodiment of the present invention provides for an immediate release of the therapeutic drug, which further improves efficacy.

The present invention is not limited to prostate cancer, and the disclosure of the present invention applies to a biomarker types and myriad corresponding probe locations, including, but not limited to brain cancer, breast cancer, colon cancer, cervical cancer, lung cancer, and the numerous forms of cancers afflicting humans, as well as non-cancer forms of abnormal biological activity detectable by biomarker sensor and similar sensors. The present invention is applicable to human, other mammal patients, as well as non-mammalian patients.

In another preferred embodiment, the unitary arrangement of a probe sensor, a communication component for the sensor and a therapeutic delivery system 200 is affixed to a cardiac stent for controlled release of drugs provided on the stent, including anticoagulants to avoid unwanted blood clotting and strokes. Additional preferred embodiments include a therapeutic response that locally releases targeted amounts of chemotherapy, and further embodiments the probe sensor 112 targets markers of Alzheimer's disease.

As shown in FIG. 4, regarding, for example, colon cancer monitoring, a preferred embodiment provides a sequential drug delivery via sequential release of chemotherapeutic agents from substrates 112a through 112d of the unitary arrangement of the probe sensor, communication component and therapeutic delivery system 200. The actual amount of chemotherapeutic agents that reach the affected area is detected by molecularly imprinted chip 113. Feedback and drug release sequence is relayed wirelessly to the external communication device 140 and monitored by a computer station acting as the external monitoring and processing device 300.

While the invention has been shown and described with reference to certain exemplary embodiments of the present invention thereof, it will be understood by those skilled in the art that various changes in form and details may be made therein without departing from the spirit and scope of the present invention as defined by the appended claims and equivalents thereof.

1. An apparatus for detection and targeted treatment of an abnormal condition, the apparatus comprising:
an external communication device;
an implantable probe for biomarker detection and wireless communication with the communication device; and
an implantable therapeutic delivery system that communicates with the external communication device,
wherein, upon biomarker change, a therapeutic agent is delivered by the delivery system.

2. The apparatus of claim 1, wherein the probe is implanted at a specific location within a patient's body corresponding to an anticipated biomarker change.

3. The apparatus of claim 1, wherein the delivery system is localized where the probe is implanted.

4. The apparatus of claim 2, wherein the probe and the delivery system are arranged on a single implant.

5. The apparatus of claim 4, wherein the implantable probe is printed onto the delivery system.

6. The apparatus of claim 1, wherein the communication is RFID communication.

7. The apparatus of claim 1, wherein an external monitoring and processing device processes information provided by the probe.

8. The apparatus of claim 1, wherein the detection probe is a nanosensor implanted at time of biopsy or surgery.

9. The apparatus of claim 2, wherein the patient is one of a human patient and a non-human patient.

10. A method of remote detection of abnormal biological activity, the method comprising:
detecting, by an implantable probe, a biomarker;
wirelessly communicating the detection to an external communication device; and
wirelessly communicating between the communication device and an implantable therapeutic delivery system,
wherein the implantable therapeutic delivery system releases a therapeutic agent based on the communication to provide targeted treatment of the abnormal biological activity.

11. The method of claim 10, wherein the probe is implanted at a specific location within a patient's body corresponding to anticipated biomarker activity to detect a change in either an amount or activity of the biomarker.

12. The method of claim 10, wherein the delivery system is localized where the probe is implanted.

13. The method of claim 11, wherein the probe and the delivery system are arranged on a single implant.

14. The method of claim 13, wherein the implantable probe is printed onto the delivery system.

15. The method of claim 10, wherein the communication is RFID communication.

16. The method of claim 10, wherein an external monitoring and processing device processes information provided by the probe.

17. The method of claim 10, wherein the detection probe is a nanosensor implanted at time of biopsy or surgery.

18. The method of claim 10, wherein the patient is one of a human patient and a non-human patient.

19. The method of claim 10, wherein implantable probe comprises an RFID tag for wireless communication.

20. An apparatus for detection and targeted treatment of an abnormal condition, the apparatus comprising:
an external communication device;
an implantable probe for detection of the abnormal condition and wireless communication with the communication device; and
an implantable therapeutic delivery system that communicates with the external communication device,
wherein, upon detection, a therapeutic agent is delivered at the site of the abnormal condition.
